(19) United States
(12) Patent Application Publication
(10) Pub. No.: US 2003/0036705 A1
(43) Pub. Date: Feb. 20, 2003

(54) Ultrasonic probe device having an impedance mismatch with rapid attachment and detachment means

(75) Inventors: Bradley A. Hare, Chelmsford, MA (US); Robert A. Rabiner, North Reading, MA (US); Kevin J. Ranucci, North Attleboro, MA (US); Rebecca I. Marciante, North Reading, MA (US); Mark J. Varady, Marlborough, MA (US); Roy M. Robertson, Ipswich, MA (US)

Correspondence Address: PALMER & DODGE, LLP, RICHARD B. SMITH, 111 HUNTINGTON AVENUE, BOSTON, MA, 02199, US

(73) Assignee: Omnisonics Medical Technologies, Inc.

(21) Appl. No.: 10/268,487

(22) Filed: Oct. 10, 2002

Related U.S. Application Data
(63) Continuation-in-part of application No. 09/975,725, filed on Oct. 11, 2001.
Application No. 09/975,725 is a continuation-in-part of application No. 09/625,803, filed on Jul. 26, 2000.
(60) Provisional application No. 60/157,824, filed on Oct. 5, 1999.

(51) Int. Cl. A61B 8/00

(52) U.S. Cl. 600/437

(57) ABSTRACT

An ultrasonic tissue ablation device comprising a transversely vibrating small-diameter probe and a coupling assembly for probe attachment and detachment that that enables the probe to disengage from the device body. The probe detachability allows for insertion, manipulation, and withdrawal independently of the device body. The probe can be used with acoustic and/or aspirations sheaths to enhance tissue ablation. The device body includes an ultrasonic energy source and a horn assembly. The probe of the present invention is engaged to the device body in a manner which creates an impedance mismatch between the probe and the device body which allows the probe and the device body to operate as separate acoustic systems. The present invention also comprises a method for the removal of vascular occlusions in a blood vessels.

### RELATED APPLICATIONS

This application is a continuation in part of U.S. application Ser. No. 09/975,725 filed on Oct. 11, 2001, which is a continuation in part of U.S. application Ser. No. 09/625,803 filed on Jul. 26, 2000 which claims priority to U.S. Provisional Application No. 60/157,824 filed on Oct. 5, 1999, the entirety of all these applications are hereby incorporated by reference.

### FIELD OF THE INVENTION

The present invention relates generally to medical devices, and more particularly to an apparatus and method for using an ultrasonic medical device having an impedance mismatch with a rapid attachment and detachment means that operates in a transverse mode which treats emulsification of endovascular materials by causing tissue fragmentation of occlusive materials.

### BACKGROUND OF THE INVENTION

Vascular occlusions (clots or thrombi and occlusional deposits, such as calcium, fatty deposits, or plaque) result in the restriction or blockage of blood flow in a vessel in which they may occur. Occlusions may result in oxygen deprivation (“ischemia”) of tissues supplied by these blood vessels. Prolonged ischemia may result in permanent damage of the tissue and may lead to myocardial infarction, stroke, or death. Targets susceptible to such occlusions include, but are not limited to, coronary arteries, peripheral arteries and other blood vessels. The disruption of an occlusion or thrombolysis can be effected by pharmacological agents and/or mechanical means.

Ultrasonic probes are devices which use ultrasonic energy to fragment body tissue (see, e.g., U.S. Pat. No. 5,112,300; U.S. Pat. No. 5,180,363; U.S. Pat. No. 4,989,583; U.S. Pat. No. 4,931,047; U.S. Pat. No. 4,922,902; and U.S. Pat. No. 3,805,787) and have been used in many surgical procedures. The use of ultrasonic energy has been proposed both to mechanically disrupt clots and to enhance the intravascular delivery of drugs to clot formations (see, e.g., U.S. Pat. No. 5,725,494; U.S. Pat. No. 5,728,062; and U.S. Pat. No. 5,735,811). Ultrasonic devices used for vascular treatments typically comprise an extra-corporeal transducer coupled to a solid metal wire that is attached to a plurality of wires. The device is then threaded through the blood vessel and placed in contact with the occlusion (see, e.g., U.S. Pat. No. 5,269,297). In some cases, the transducer is delivered to the site of the clot, the transducer comprising a bendable plate (see, U.S. Pat. No. 5,931,805).

The ultrasonic energy produced by an ultrasonic probe is in the form of very intense, high frequency sound vibrations that result in powerful chemical and physical reactions in the water molecules within a body tissue or surrounding fluids in proximity to the probe. These reactions ultimately result in a process called “cavitation,” which can be thought of as a form of cold (i.e., non-thermal) boiling of the water in the body tissue, such that microscopic bubbles are rapidly created and destroyed in the water creating cavities in their wake. As surrounding water molecules rush in to fill the cavity created by collapsed bubbles, they collide with each other with great force. Cavitation results in shock waves running outward from the collapsed bubbles which can fragment or ablate material such as surrounding tissue in the vicinity of the probe.

Some ultrasonic probes include a mechanism for irrigating an area where the ultrasonic treatment is being performed (e.g., a body cavity or lumen) in order to wash debris away from the area. Mechanisms used for irrigation or aspiration described in the art are generally structured such that they increase the overall cross-sectional profile of the probe. The overall cross-sectional profile of the probe is increased by including inner and outer concentric lumens within the probe to provide irrigation and aspiration channels for removal of debris. Prior art probes also maintain a strict orientation of the aspiration and the irrigation mechanism, such that the inner and outer lumens for irrigation and aspiration remain in a fixed position relative to one another. Thus, the irrigation lumen does not extend beyond the suction lumen (i.e., there is no movement of the lumens relative to one another) and any aspiration is limited to picking up fluid and/or tissue remnants within the defined area between the two lumens.

An additional drawback of existing ultrasonic medical probes is that they typically remove tissue relatively slowly in comparison to instruments that excise tissue by mechanical cutting. Part of the reason for this is that existing ultrasonic devices rely on a longitudinal vibration of the tip of the probe for their tissue-disrupting effects. Because the tip of the probe is vibrated in a direction in line with the longitudinal axis of the probe, a tissue-destroying effect is only generated at the tip of the probe. One solution that has been proposed is to vibrate the tip of the probe in a direction perpendicular to the longitudinal axis of the probe in addition to vibrating the tip in the longitudinal direction. It is proposed that such motions will supplement the main point of tissue destruction, which is at the probe tip, since efficiency is determined by the surface area of the probe tip.

The longitudinal probe vibration required for tissue ablation in prior art devices necessitates that the probe lengths be relatively short. The use of a long probe may result in a substantial loss of ultrasonic energy at the probe tip due to thermal dissipation and undesirable horizontal vibration that may interfere with the required longitudinal vibration.

A large diameter probe cannot negotiate the anatomical curves of tubular arterial and venous vessels due to the probe's inflexibility, and the large diameter probe may cause damage to the vessels. Although a narrow probe diameter is advantageous for negotiation through narrow blood vessels and occluded arteries, the utilization of such a probe has been precluded by an inability to effectively control the vibrational amplitude of a small diameter probe, resulting in potential damage to the probe and a substantial risk of tissue damage resulting from the probe's use. The use of a narrow diameter probe has been disclosed in the art for providing greater maneuverability and ease of insertion into narrow diameter blood vessels.

The relatively high-energy requirement for prior art ultrasonic probes causes probe heating that can cause fibrin to re-clot blood within the occluded vessel (thermally induced re-occlusion). Additionally, the elevation in probe temperature is not just limited to the probe tip, but also occurs at points wherein the small diameter probes have to bend to conform to the shape of the blood vessel.

Prior art ultrasonic probes used in endovascular procedures are attached to an energy source (i.e., by welding) thereby precluding probe detachment from the energy source. Moreover, such devices utilizing longitudinal vibration require a proximal contact with the transducer or the probe handle segment in order to prevent a “hammering” effect that can result in probe damage.

The limitations surrounding the use of a narrow diameter probe has precluded the use of ultrasonic tissue ablation devices in surgical procedures where access to a vascular occlusion requires traversing a lengthy or sharply curved path along tubular vessels. The self-suggesting idea of effecting ultrasonic transmission through a plurality of flexible thin wires has been found impracticable because (1) relatively high power (˜25 watts) is required to deliver sufficient energy to the probe tip, and (2) such thin wires tend to perform buckling vibrations, resulting in almost the entire ultrasonic power provided to the probe being dissipated during its passage to the probe tip.

Based on the aforementioned limitations of prior art ultrasonic probes, there is a need for an ultrasonic probe functioning in a transverse mode that overcomes limitations imposed by the use of a narrow diameter probe in the area of rapid tissue ablation. Such limitations include the need to predict the frequency of the probe in operation.

A further limitation encountered when attempting to operate a narrow, ultrasonic probe has been anticipating and calculating the large deviations in the frequency of the vibration of the probe when the probe is in use. As is known in the art, a probe will only resonate when the frequency of the probe matches the frequency of the energy being supplied to the probe.

In electricity, impedance is measured in ohms. Impedance is the degree to which an electric circuit resists the flow of electric current when a voltage is impressed across its terminals. Impedance is expressed as the ratio of the voltage impressed across a pair of terminals to the current flow between those terminals. When an electrical circuit is supplied with a steady direct current, the impedance equals the total resistance of the circuit. The resistance depends upon the number of electrons that are free to become part of the electrical current and upon the difficulty that the electrons have in moving through the circuit. When a circuit is supplied with alternating current, the impedance is affected by the inductance and capacitance in the circuit. When supplied with alternating electrical current, elements of the circuit that contain inductance or capacitance build up voltages that act in opposition to the flow of current. This opposition is called reactance, and it must be combined with the resistance to find the impedance. The reactance produced by inductance is proportional to the frequency of the alternating current. The reactance produced by capacitance is inversely proportional to the frequency of the alternating current.

In order for a source of electricity that has an internal impedance to transfer maximum power to a device that also has an impedance, the two impedance must be matched. For example, in the simple case of pure resistances, the resistance of the source must also equal the resistance of the device. Impedance matching is important in any electrical or electronic system in which power transfer must be maximized.

Medical applications requiring the use of ultrasonic energy often require transmission of the energy into locations deep within the body. The device will often have to traverse a tortuous and unpredictable path. The necessary twisting and bending of the delivery mechanism will create large and unpredictable changes in the static stresses acting on the device, which in turn will cause the resonant frequencies for ultrasonic vibration to vary making it difficult to maintain vibration. As such, the source of ultrasonic energy can not be set at a known frequency that matches the frequency of the probe. Such problems have led to extremely complex electronic systems attempting to match the frequency of the probe and the frequency of the ultrasonic energy source. The prior art devices have not adequately matched the impedance of separate elements of an ultrasonic system.

U.S. Pat. No. 5,974,884 to Sano et al. discloses an ultrasonic diagnostic apparatus which comprises a probe which has a transducer for transmitting and receiving ultrasonic waves and an acoustic matching layer in which the acoustic impedance thereof is varied continuously in the thickness direction. This prevents a discontinuity in the acoustic impedance, thus giving rise to less reflection of the ultrasonic wave within the acoustic matching layer. The prior art teaches a device for matching the impedance of a drive system to a delivery system in order to increase efficiency.

U.S. Pat. No. 5,434,827 to Bolorforosh discloses an ultrasonic system which provides an impedance match between a probe and a medium under examination by the probe. The Bolorforosh probe employs one or more piezoelectric ceramic elements. Each element has a respective front face and a respective piezoelectric ceramic layer integral therewith for substantially providing a desired acoustic impedance match between the bulk acoustic impedance element and an acoustic impedance of the medium under examination. By providing the acoustic impedance match, the inert piezoelectric layer helps to provide efficient acoustic coupling between the probe and the medium under examination by the probe. The prior art teaches a device for matching the impedance of a drive system to a delivery system in order to increase efficiency.

U.S. Pat. No. 4,523,122 to Tone et al. discloses an ultrasonic transducer which comprises an acoustic impedance-matching layer or layers having an optimum acoustic impedance for achieving a match between a piezoelectric transducer or magneto-striction element and air. Tone et al. provides an ultrasonic transducer which comprises a specific combination of two acoustic impedance-matching layers having specific ranges of acoustic impedance, respectively, whereby ultrasound signals of good pulse response characteristic are transmittable in high efficiency and receivable in high sensitivity over a wide range of high frequency. The prior art teaches a device for matching the impedance of a drive system to a delivery system in order to increase efficiency.

Prior art devices and methods of controlling the frequency of an ultrasonic probe are complicated and involve complex electronics. As discussed above, prior art devices and methods also involve various attempts to match the impedance of the probe to the driving system. Therefore, there is a continuing need in the art for further developments in the area of controlling and maintaining the frequency of an ultrasonic probe. In particular, a simple, inexpensive apparatus and method which would allow an ultrasonic probe having an impedance mismatch and a quick attachment and detachment means to resonate in a transverse mode at a determined frequency is needed in the art.

### SUMMARY OF THE INVENTION

The present invention is an apparatus emitting ultrasonic energy in a transverse mode used in combination with an elongated flexible probe, wherein the probe is rapidly attachable to and detachable from the ultrasonic energy source component of the device. The probe of the present invention vibrates substantially in a direction transverse to the longitudinal axis of the probe and is capable of emulsifying endovascular materials, particularly tissue. The diameter of the probe is sufficiently small to confer flexibility on the probe so as to enable negotiation of the probe through narrow and anatomically curved tubular vessels to the site of an occlusion. The probe of the present invention is designed to work in conjunction with standard vascular introducers and guide catheters.

Another aspect of the present invention is to provide a rapidly attachable and detachable or “quick attachment-detachment” means (referred to hereinafter as “QAD”) attaching/detaching the ultrasonic probe to and from the ultrasonic energy source, thereby enabling manipulation and positioning of the probe within the body vessel without being limited by relatively bulky energy source. In addition, the present invention provides an ultrasonic device which comprises two acoustically separate components, a drive system and a delivery mechanism. Acoustically separate components allow an ultrasonic energy source (i.e., a horn) to act at a pre-determined and nearly constant frequency despite large and unpredictable changes in the frequency of a delivery mechanism (i.e., a probe).

The present invention provides an ultrasonic device in which the probe and the energy source are acoustically separate components. By establishing an impedance mismatch between a drive system (i.e., the energy source) and a delivery mechanism (i.e., the probe), the drive system may be allowed to operate at a fixed, pre-determined frequency despite rapid and unpredictable changes in the frequency of the delivery mechanism.

Additionally, the probe of the present invention comprises a concentric, tubular sheath to facilitate fluid irrigation, aspiration of ablated tissue fragments and the introduction of a therapeutic drug to a treatment site.

In general, it is an object of the present invention to provide an ultrasonic medical device for removing vascular occlusions comprising a detachable elongated catheter compatible guide wire probe capable of vibrating in a transverse mode.

Additionally, the present invention provides a method to treat vascular occlusions with an ultrasonic device having an impedance mismatch and a quick attachment and detachment means.

Additional objects and features of the present invention will become apparent from the following description, in which the preferred embodiments are set forth in detail in conjunction with the accompanying drawings.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a general view of the elongated flexible wire probe catheter of the invention.

FIG. 2A shows a varied diameter probe, QAD collet-horn assembly and locking nut disassembled.

FIG. 2B show a varied diameter probe, QAD collet-horn assembly and locking nut assembled.

FIG. 2C shows an assembled configuration of a uniformly small diameter wire probe.

FIG. 3 shows a cross sectional view of the probe assembled to QAD collet assembly.

FIG. 4A shows the locking nut viewed from a first cylindrical end.

FIG. 4B shows the locking nut from a second cylindrical end.

FIG. 5 shows a cross sectional view of the locking nut coupling the probe to the QAD collet-horn assembly.

FIG. 6 shows the threaded horn component of the QAD collet-horn assembly.

FIG. 7 shows scaled and cross-sectional views of an embodiment of the QAD collet assembly.

FIG. 8A shows a first view of an embodiment of the QAD collet rod and housing assembly.

FIG. 8B shows a second view of an embodiment of the QAD collet rod and housing assembly.

FIG. 9 shows scaled and cross-sectional views of an embodiment of the QAD collet assembly.

FIG. 10A shows a first view of an embodiment of the QAD collet rod and housing assembly.

FIG. 10B shows a second view of a embodiment of the QAD collet rod and housing assembly.

FIG. 11 shows scaled and cross-sectional views of an embodiment of the QAD collet assembly.

FIG. 12A shows a first view of an embodiment of a collet, a QAD base component and a compression housing.

FIG. 12B shows a second view of an embodiment of the collet, the QAD base component and the compression housing.

FIG. 12C shows a third view of an embodiment of the collet, the QAD base component and the compression housing.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION OF THE INVENTION

The present invention is an ultrasonic tissue ablation device comprising a transversely vibrating elongated probe, and a coupling assembly for probe attachment and detachment that enables the probe assembly and separation from a device body that includes the ultrasound energy source and a sound conductor. The present invention also comprises a method of use for removal of vascular occlusions in blood vessels. The coupling assembly enables incorporation of elongated probes with small cross sectional lumens such as a catheter guidewires. The probe detachability allows insertion, manipulation and withdrawal of the probe independently of the device body.

The probe can be used with acoustic and/or aspirations sheaths to enhance destruction and removal of an occlusion. The horn assembly of the device that contains a sound conducting horn functions as an energy regulator and reservoir for the probe, and precludes loss of probe cavitation energy by its bending or damping within the blood vessel.

The present invention provides an ultrasonic device in which the probe and the energy source are acoustically separate components. By establishing an impedance mismatch between a drive system (i.e., the energy source) and a delivery mechanism (i.e., the probe), the drive system may be allowed to operate at a fixed, predetermined frequency despite rapid and unpredictable changes in the frequency of the delivery mechanism.

The following terms and definitions are used herein:

“Anti-node” as used herein refers to a region of maximum energy emitted by an ultrasonic probe at or proximal to a specific location along the longitudinal axis probe.

“Cavitation” as used herein refers to shock waves produced by ultrasonic vibration, wherein the vibration creates a plurality of microscopic bubbles which rapidly collapse, resulting in a molecular collision by water molecules which collide with force thereby producing the shock waves.

“Fenestration” as used herein refers to an aperture, window, opening, hole, or space.

“Impedance” as used herein refers to a measure of a physical system to an applied force. Mathematically, the acoustic impedance is defined as F/v, where F is the applied force and v is the velocity of the material. For the specific case of a plane longitudinal wave the acoustic impedance(Z) is defined by the equation Z=ρcA, where p is the density, c is the speed of sound of the material and A is the cross sectional area with normal parallel to the direction of wave propagation. For other modes of propagation, the impedance can be determined from the definition using the appropriate equation of motion with similar results.

“Node” as used herein refers to a region of minimum energy emitted by an ultrasonic probe at or proximal to a specific location along the longitudinal axis probe.

“Sheath” as used herein refers to a device for covering, encasing, or shielding, in whole or in part, a probe or a portion thereof and the sheath is connected to an ultrasonic generation means.

“Transverse” as used herein refers to vibration of a probe not parallel to the longitudinal axis of the probe. A “transverse wave” as used herein is a wave propagated along an ultrasonic probe in which the direction of the disturbance at each point of the medium is perpendicular to the wave vector.

“Tuning” as used herein refers to a process of adjusting the frequency of the ultrasonic generator means to select a frequency that establishes a standing wave along the length of the probe.

“Ultrasonic probe” as used herein refers to any medical device utilizing ultrasonic energy with the ability to ablate debris including, but not limited to, probes, elongated wires, and similar devices known to those skilled in the art. The ultrasonic energy of the ultrasonic probe may be in either a longitudinal mode or a transverse mode.

The present invention provides an ultrasonic device operating in a transverse mode for removing a vascular occlusion by causing fragmentation of occlusive materials, such as tissue. Because the device is minimally invasive and flexible, it can be inserted into narrow, tortuous blood vessels without risking damage to those vessels.

Transverse vibration of the probe in such a device generates multiple anti-nodes of cavitational energy along the longitudinal axis of the probe, which are resolved into caviational anti-nodes emanating radially at specific points along the probe. Transversely vibrating ultrasonic probes for tissue ablation are described in the Assignee's co-pending applications U.S. application Ser. No. 09/975,725; U.S. application Ser. No. 09/618,352; U.S. application Ser. No. 09/917,471; and U.S. application Ser. No. 09/776,015 which further describe the design parameters for such a probe used in an ultrasonic devices for tissue ablation. The entirety of these applications are hereby incorporated by reference.

The occlusive material is fragmented into debris in the range of sub-micron sizes. The transverse vibrations generate a retrograde flow of debris that carries the debris away from the probe tip. A transverse mode of vibration of the ultrasonic probe according to the present invention differs from a conventional axial (or longitudinal) mode of vibration. Rather than vibrating in the axial direction, the probe vibrates substantially in a direction transverse (perpendicular) to the axial direction. As a consequence of the transverse vibration of the probe, the tissue-destroying effect of the device is not limited to the region coming into contact with the tip of the probe. Rather, as an active portion of the probe is positioned in proximity to an occlusion or other blockage of a blood vessel, the tissue is removed in all areas adjacent to the plurality of anti-nodes that are produced along the entire length of the active section of the probe and the area of treatment extends approximately 6 mm around the probe.

By allowing transverse vibration, the present invention is capable of fragmentation of larger areas of tissue spanning the entire length of the active section of the probe as opposed to only treating tissue at the probe tip. The tissue is treated by the generation of a plurality of anti-nodes along the entire length of the active section of the probe. Since substantially larger affected areas within an occluded blood vessel can be denuded of the occlusive tissue in a short time, actual treatment time is greatly reduced by using the ultrasonic device of the present invention.

A distinguishing feature of the present invention is the ability to utilize probes of extremely small diameter (approximately 0.025 inches and smaller) without a loss of efficiency when compared to prior art devices. A small diameter device of the present invention does not result in a decreased efficiency as compared to a large diameter probe as found in the prior art because the tissue fragmentation process is not dependent on the area of the probe tip (the distal end). Highly flexible probes can therefore, be designed to mimic device shapes enabling insertion into a highly occluded or extremely narrow interstice within a blood vessel without resulting in breakage of the probe or puncture or damage of the tissue or body cavity while ensuring optimal results.

Another distinguishing feature of a small diameter probe of the present invention is that the probe diameter is approximately the same over their entire length. In a preferred embodiment, the probe diameter at the proximal end is about 0.025 inches and the probe diameter at the distal end is about 0.015 inches, so the probe is adaptable to standard vascular introducers. Since the rear segment (proximal end) of the probe does not have a non-cylindrical shape or “bulk”, catheters and guides can be introduced over the ends of the elongated wire probe of the invention, thereby allowing their use in standard-configuration endovascular procedures.

Another advantage provided by the present invention is its ability to rapidly remove occlusive material from large areas within cylindrical or tubular regions including, but not limited to, arteries and arterial valves or selected areas within the tubular walls, which has not been possible with the use of previously disclosed devices that rely on the longitudinal vibrating probe tip for effecting tissue fragmentation.

The number of anti-nodes occurring along the axial length of the probe is controlled by changing the frequency of energy supplied by the ultrasonic generator. The exact frequency, however, is not critical and a ultrasonic generator run at, for example, 20 kHz is generally sufficient to create an effective number of tissue destroying anti-nodes along the axial length of the probe. The present invention allows for selective tissue treatment because the ultrasonic device transmits energy across a frequency range of about 20 kHz to about 80 kHz. The amount of ultrasonic energy to be supplied to a particular treatment site is a function of the amplitude and frequency of vibration of the probe. In general, the amplitude is in the range of about 25 microns to about 250 microns, and the frequency in the range of about 20,000 to about 80,000 Hertz (20-80 kHz). In the currently preferred embodiment, the frequency of ultrasonic energy is from about 20,000 Hertz to about 35,000 Hertz (20-35 kHz). Frequencies in this range are specifically destructive of hydrated (water-laden) tissue and other vascular occlusive material, while substantially ineffective toward high-collagen connective tissue, or other fibrous tissues including, but not limited to, vascular tissue and skin or muscle tissue.

In a preferred embodiment of the present invention, the ultrasonic device comprises an ultrasonic generator that is coupled to a probe having a proximal end and a distal end. In one embodiment, a magneto-strictive generator may be used for the generation of ultrasonic energy. In a preferred embodiment, the generator is a piezoelectric transducer that is mechanically coupled to the probe enabling a transfer of ultrasonic excitation energy and causing the probe to oscillate in a transverse direction relative to its longitudinal axis. The device is designed to have a small cross-sectional profile allowing the probe to flex along its length, thereby allowing it to be used in a minimally invasive manner. Transverse oscillation of the probe generates a plurality of anti-nodes along the longitudinal axis of the member, thereby efficiently destroying an occlusion located in the proximity of the active length of the probe. A significant feature of the invention is the retrograde movement of debris that results from the transversely generated energy. The debris may be moved away from the tip of the probe and backwards up along the shaft of the probe. The amount of ultrasonic energy applied to a particular treatment site is a function of the amplitude and frequency of vibration of the probe, the longitudinal length of the probe, the proximity of the probe to a tissue, and the degree to which the probe is exposed to a tissue.

The ultrasonic device of the invention comprises a longitudinal resonator including, but not limited to, a Mason (Langevin) horn that is in contact with an elongated catheter wire probe through a coupling assembly. The horn assembly is in turn, coupled to an ultrasound energy source. Upon device activation, ultrasonic energy from the source is transmitted to the horn assembly wherein it is amplified by the horn and in turn, transmitted to the probe through the coupling assembly. Transverse vibrational modes along the longitudinal axis of the probe that are coupled to the horn resonance will be excited upon the delivery of ultrasonic energy to the probe.

A limitation that has been encountered when attempting to operate a small-diameter ultrasonic probe in a transverse mode has been anticipating and calculating the large deviations in the frequency of the vibration of the probe when the probe is in use. As is known in the art, a probe will only resonate when the frequency of the probe matches the frequency of the energy being supplied to the probe.

In order for a source of energy that has an internal impedance to transfer maximum power to a device that also has an impedance, the two impedances must be matched. For example, in the simple case of pure resistances, the resistance of the source must also equal the resistance of the device. Impedance matching is important in any electrical or electronic system in which power transfer must be maximized.

Medical applications requiring the use of ultrasonic energy often require transmission of the energy into locations deep within the body. The device will often have to traverse a tortuous and unpredictable path. The necessary twisting and bending of the delivery mechanism will create large and unpredictable changes in the static stresses acting on the device which will cause the resonant frequencies for ultrasonic vibration to vary making it difficult to maintain vibration. As such, the source of ultrasonic energy can not be set at a known frequency that matches the frequency of the probe.

The present invention separates the ultrasonic medical device into two loosely coupled vibrating systems: a delivery mechanism responsible for the delivery of the vibrations (i.e., a probe); and a drive system responsible for maintaining the vibration (i.e., an energy source).

Ultrasonic vibrations will be produced in the probe whenever a mechanical resonance of the probe can be coupled to the vibration of the drive system. In a preferred embodiment of the present invention, a mechanical resonance of the probe is coupled to the vibration of the drive system by using a longitudinal mode drive system to induce a buckling in the probe thereby inducing a transverse vibration in the probe. In another embodiment of the present invention, a transverse mode drive system is used to induce a transverse mode directly. Sustained transverse vibration of the probe will occur whenever the resonant frequency of a transverse vibration in the probe is coupled with the frequency of the drive system.

In a preferred embodiment of the present invention, the probe is a long, flexible wire. The drive system is a typical longitudinal horn of the Mason (Langevin) type operating in a longitudinal mode. In one embodiment, the Mason horn is a one-half wavelength long, with a one-quarter wavelength in the back for a transducer, a one-quarter wavelength in the front leading to the attachment point to the probe, and a middle which is located at a node. In a preferred embodiment, a length of the horn approximates an integer multiple of one-half wavelength of a vibration.

In one embodiment of the present invention, the horn comprises aluminum. In one embodiment of the present invention, the horn comprises an aluminum alloy. In one embodiment, the horn of the present invention comprises steel. In one embodiment of the present invention, the horn comprises a ferrous material. Those of skill in the art will recognize that the horn could be composed of other material within the spirit and scope of the invention.

In one embodiment, the probe is of a sufficiently low stiffness (a thin wire) that the distance between two successive anti-nodes will be very close. In one embodiment, the wire is approximately 0.020 inches in diameter and the spacing between the transverse modes will be approximately 200 Hz.

External forces acting on the wire will cause the modes to shift frequency rapidly. When the probe is deployed into a tight bend, shifts in the resonant frequency may be as much as 1000 Hz. In one embodiment of the present invention, a longitudinal drive system is operated at moderate drive levels and vibration can be sustained over at least 200 Hz of tuning. It is therefore likely that there will always be a transverse resonance coupled to the driving frequency to sustain vibration on the probe.

In the present invention, the maintenance of vibrations on the probe depends only on the maintenance of vibration in the drive system. If the vibrations on the wire are strongly coupled back to the drive system, traditional means of detecting and stabilizing the drive system resonance including, but not limited to, microphone transducers and current-voltage phase detection, will be unable to distinguish the transverse vibrations from the drive system vibration. The present invention overcomes this limitation of the prior art devices by de-coupling the two systems.

Sound travels through materials under the influence of sound pressure. Because molecules or atoms of a solid are bound elastically to one another, the excess pressure results in a wave propagating through the solid.

The acoustic impedance (Z) of a material is defined as the product of the density (ρ), the speed of sound (c), and the cross sectional area (A) of the material by the following equation: Z=ρcA. Acoustic impedance is important in: (1) the determination of acoustic transmission and reflection at the boundary of two materials having different acoustic impedance; (2) the design of ultrasonic transducers; and (3) assessing absorption of sound in a medium.

Ultrasonic waves are reflected at boundaries where there are discontinuities in acoustic impedance (Z). This is commonly referred to as impedance mismatch. The fraction of the incident-wave intensity in the reflected waves can be derived because the particle velocity and local particle pressures are required to be continuous across the boundary between materials.

Vibrations traveling outward from the drive system will be reflected back into the drive system if they encounter a discontinuity in the mechanical impedance along the way. The mechanical impedance is defined as the ratio of the driving force to the velocity at an interface. For two bars of different diameters attached to one another (or machined from a single bar), there will be a discontinuity at the point of attachment. If the bars are of a significantly different diameter, a small amount of energy will be coupled into the second bar from the first bar.

In a preferred embodiment of the present invention, a discontinuity is placed at the point of connection between the probe and the drive system. The discontinuity will cause some of the outgoing energy to be reflected back into the drive system. The amount of energy reflected back into the drive system will depend on the extent of the discontinuity. In one embodiment of the present invention, approximately 80% of the energy is reflected back into the horn and 20% of the energy is transferred to the probe. In a preferred embodiment of the present invention, the discontinuity is created through a large change in the cross sectional area at the point of attachment. In one embodiment of the present invention, the discontinuity is created by a change in the material properties at the attachment point between the horn and the probe causing a large change in the speed of sound at the attachment point. In one embodiment of the present invention, the discontinuity is created by a change in the material properties causing a large change in the density of the materials used to create the attachment.

In a preferred embodiment of the present invention, the discontinuity is ideally located at a location which corresponds to an anti-node of the drive system vibration. At the discontinuity, reflections will return to the drive system in phase and the location of the discontinuity can be used to determine the resonant frequency of the drive system. In one embodiment of the present invention, the location of the discontinuity is at a node. If the attachment point is at a node, the device would require increasing the length of the horn by placing a second discontinuity placed about one-fourth wavelength away from the first discontinuity to cancel the reflection going back to the drive system.

The coupling between the probe and the horn is adjusted so as to present a discontinuity with a relatively large impedance mismatch. In a preferred embodiment of the present invention, the discontinuity is located at an anti-node of the horn. Longitudinal waves impinging on the coupling interface are either reflected back into the horn or transmitted out to the probe in proportion to the degree of impedance mismatch at the discontinuity point. The greater the degree of impedance mismatch, the less energy is transmitted out to the probe. In a preferred embodiment, the coupling interface is configured in a manner so as to reflect most of the energy back into the horn. The horn, therefore, essentially acts as an energy storage device or “reservoir”, thereby allowing a substantial increase in drive amplitude.

Since the energy coupled into the elongated probe is a small portion of the energy reflected back to the horn, changes in the transverse oscillation on the probe due to bending or damping have minimal effect on the longitudinal resonance of the horn. By decoupling the transverse probe oscillation from the longitudinal horn resonance, the electrical source of the vibrations (piezoelectric or magnetostrictive) compensate only for shifts in the resonant frequency of the horn (due to temperature, manufacturing variations, etc.). The drive mechanism is, therefore, independent of vibrational motion of the probe.

For a longitudinal plane wave incident on the interface between two materials of different impedances the percentage of energy reflected (R) and the percent age of energy transmitted (T) are defined as:

$$\begin{matrix}R = \frac{{( {Z}_{1} - {Z}_{2} )}^{2}}{{( {Z}_{1} + {Z}_{2} )}^{2}} & [ 1.1 ] \\ T = \frac{4 {Z}_{1} {Z}_{2}}{{( {Z}_{1} + {Z}_{2} )}^{2}} & [ 1.2 ]\end{matrix}$$

Consider the special case where the material is the same on each side of the interface, but the cross sectional areas differ. The reflection and transmission coefficients become:

$$\begin{matrix}R = \frac{{( {A}_{1} - {A}_{2} )}^{2}}{{( {A}_{1} + {A}_{2} )}^{2}} & [ 1.3 ] \\ T = \frac{4 {( ρ c )}^{2} ( {A}_{1} {A}_{2} )}{{( ρ c )}^{2} {( {A}_{1} + {A}_{2} )}^{2}} = \frac{4 {A}_{1} {A}_{2}}{{( {A}_{1} + {A}_{2} )}^{2}} & [ 1.4 ]\end{matrix}$$

A typical example with diameters Ø₁=0.186 inches and Ø₂=0.025 inches on each side of the interface gives an area relation between the two sides of

$$\frac{{A}_{1}}{{A}_{2}} = \frac{π {r}^{2}}{π {r}^{2}} , {A}_{1} = 50 {A}_{2} ( t y p i cal ) .$$

From equation [1.3],

$$R = \frac{{49}^{2}}{{51}^{2}} = .92$$

and equation [1.4],

$$T = \frac{200 {A}_{2}^{2}}{{51}^{2} {A}_{2}^{2}} = .08$$

As shown in the above equations, 92% of an incident plane wave would be reflected and 8% would be transmitted.

An additional advantage of the present invention over the prior art is that the transverse vibrating elongated probe of the invention does not require its terminal end be permanently affixed to the horn assembly, since a “hammering” action associated with longitudinal vibration is absent. The elongated probe of the invention can therefore be coupled, and not welded, to the horn via a coupling assembly that engages the probe along the cylindrical surface near its terminal end in a non-permanent way. The coupling assembly of the invention therefore, allows for quick attachment and detachment of the probe from the horn assembly and the source components, thereby enabling manipulation of the elongated flexible probe into anatomically curved blood vessels without hindrance by a bulky horn and energy source components. The probe of the present invention can therefore be inserted into a venal cavity and positioned near the occlusion site prior to coupling the probe to the horn source assembly. The device is then activated to effect tissue ablation and removal, after which the probe is decoupled from the horn and source component for an easy removal of the probe from the cavity.

In a preferred embodiment of the present invention, a longitudinal horn is coupled to an elongated wire catheter by a coupling assembly that is rapidly attachable and detachable. In a preferred embodiment, the coupling assembly comprises a quick attachment-detachment (QAD) collet. The attachment of the coupling assembly to the elongated probe is located at an anti-node of the horn and the dimensions are scaled (i.e., the collet head has a relatively larger diameter at the attachment point than the diameter of the probe) to produce an optimal impedance mismatch (as discussed above.). In another embodiment, the attachment of the coupling assembly to the elongated probe is located at a node. In an embodiment of the invention, the elongated probe is permanently attached to the coupling assembly.

The QAD collet of the invention is housed within an externally mounted compressive clamp that is capable of exerting a compressive force on the collet after insertion of the ultrasonic probe into the collet, thereby causing a non-removable attachment of the probe to the coupling assembly. The collet therefore, applies a restraining inwardly compressive force on the probe in a manner so as to not torque, twist or damage the probe. As a result, the probe can be subject to multiple attachment and detachment procedures without causing probe destruction, thereby enabling its extended reuse in surgical procedures.

In one embodiment, the collet of the present invention comprises at least one slit in its terminal compressible segment. In another embodiment, the collet comprises a plurality of slits. In a preferred embodiment of the present invention, the collet, the compressive clamp and the housing assembly are all attached to the device handle by a mechanical assembly means, such as for example, a screw thread comprising a locking nut, bayonet mount, keyless chuck and cam fittings. Alternatively, the rear segment of the mechanical assembly means is a hollow cylindrical segment comprising a screw thread that allows insertion and attachment of the ultrasonic device handle containing a drive assembly and a complementary thread arrangement to be inserted into and non-removably attached to said cylindrical segment by applying a torque. In a preferred embodiment, an ultrasonic probe is mounted to the attachment means such that the collet holds the probe at a point greater than about 1 mm and less than about 30 mm from the probe's terminal end in order to optimize the probe's vibration based on the frequency of the ultrasound transducer in the device handle.

In a preferred embodiment, the probe attachment means comprising the external compressive clamp, the collet and the collet housing are all attached to the operating handle of the ultrasonic device.

In a preferred embodiment of the present invention, the collet is retained within the confines of an outer shell that is attached to the collet housing segment of the probe attachment means in order to prevent its disassembly, thereby preventing either loss or disengagement of the collet. By an application of a torque, the outer shell compresses the collet so that the collet engages the probe. Application of such a torque causes the probe to be attached to the collet in a non-removable manner. An inner bias is maintained within the rear portion of the attachment means such that a portion of the probe protruding from the proximal end of the collet maintains contact with the surface of the collet housing within the coupling assembly.

The terminal end of the collet is tapered so as to allow the collet to maintain a true axial orientation within the coupling assembly, thereby enabling a plurality of insertions and retractions of the probe into and from the collet prior to and after device use, without causing damage to the probe. Additionally, the shape of the proximal end of the segment (a rear segment with respect to the entering probe), is designed to maximize a contact area between the collet and the distal end of the transducer-sound conductor assembly (the “drive assembly”). Upon probe attachment to the collet within the housing assembly, the collet's proximal end is shaped in any suitable form which provides maximal contact area, including, but not limited to, conical, frusto-conical, triangular, square, oblong, and ovoid. The housing assembly maintains intimate contact with the drive assembly. The four component assembly (a probe, an outer ring, a collet and a rear drive assembly) form a unitary component while the device is in operation in order to transmit sound energy from the transducer in the drive assembly to the probe without thermal or mechanical energy loss. A collet of the present invention can be designed to accommodate a range of probe diameters, or for a specific probe diameter by varying the inner diameter of the cylindrical slot. An outer diameter of the collet remains unchanged allowing attachment of probes of differing diameters into a universal coupling and drive assembly.

In one embodiment of the present invention, the elongated probe is a single diameter wire with an approximately uniform cross section offering flexural stiffness along its entire length. In one embodiment, the elongated probe is tapered or stepped along its length to control the amplitude of a transverse wave along the probe's longitudinal axis. Alternatively, the probe can be cross-sectionally non-cylindrical and capable of providing both flexural stiffness and support energy conversion along its entire length.

In a preferred embodiment, the elongated probe of the invention is chosen to be from about 30 cm to about 300 cm in length. In a preferred embodiment, the elongated probe of the invention has a length of about 70 cm to about 210 cm in length. Suitable probe materials include metallic materials and metallic alloys suited for ultrasound energy transmission. In a preferred embodiment, the metallic material comprising the elongated probe is titanium. In other embodiments, the probe is composed of a titanium alloy.

In a preferred embodiment, the elongated probe of the invention is enclosed in a sheath that provides a conduit for an irrigation fluid, provides aspiration of fragmented tissue, or delivers a therapeutic drug to an occlusion site. The sheath can extend either partially or can extend over the entirety of the probe. In addition, the probe may comprise a plurality of fenestrations for directing ultrasonic energy from the probe at specific locations within a venal cavity for selective ablation of tissue. An ultrasonic tissue ablation device comprising a sheath for removal of occlusions in blood vessels has been disclosed in assignee's co-pending application Ser. No. 09/776,015, the entirety of which is hereby incorporated by reference.

In one embodiment of the present invention, the small-diameter probe is comprised of a proximal end and a distal end with respect to the horn assembly, and is in the form of an elongated, small diameter wire incorporating a series of telescoping segments along its longitudinal axis. The probe is constructed such that the largest diameter segment is proximal to the horn assembly, and either continually or incrementally decreases in diameter from the proximal end to the distal end. As shown in the figures displaying the probe, the coupling assembly and horn assembly, the proximal end of each component refers to the end farthest from the probe tip, while distal end refers to the end closest to the probe tip.

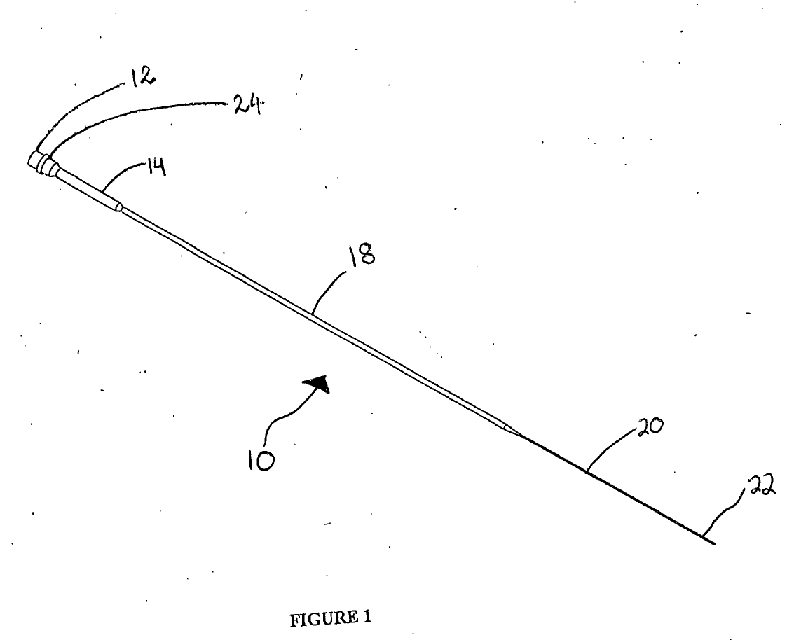

In another embodiment, the elongated probe is comprised of a constant, uniformly small-diameter wire. As displayed in FIG. 1, a preferred embodiment of the elongated ultrasonic probe 10 of the present invention comprises a proximal end 12 and a distal end 22. The probe 10 is coupled to a transducer and sound conductor assembly (not shown). The transducer and the sound conductor assembly function as a generation and a transmission source respectively, of ultrasonic energy for activation of the probe 10. The generation source may or may not be a physical part of the device itself. The probe 10 transmits ultrasonic energy received from the sound conductor along its length, and is capable of engaging the sound conductor component at its proximal end 12 via a coupling assembly with sufficient restraint to form an acoustical mass that can propagate the ultrasonic energy provided by the source.

In one embodiment, the probe diameter decreases at defined segment intervals 14, 18, and 20. Segment 20 because of its small diameter, is capable of flexing more than segments 14 and 18, thereby enabling the probe 10 to generate more cavitation energy along segment 20 at the distal end 22 as opposed to those segments at the proximal end of the probe 10. Energy from the generator is transmitted along the length of the probe 10 causing the probe 10 to vibrate in a direction that is transverse to its longitudinal axis. Probe interval 14 has a head segment 24 for engaging the coupling assembly for attachment to the sound conductor-transducer assembly.

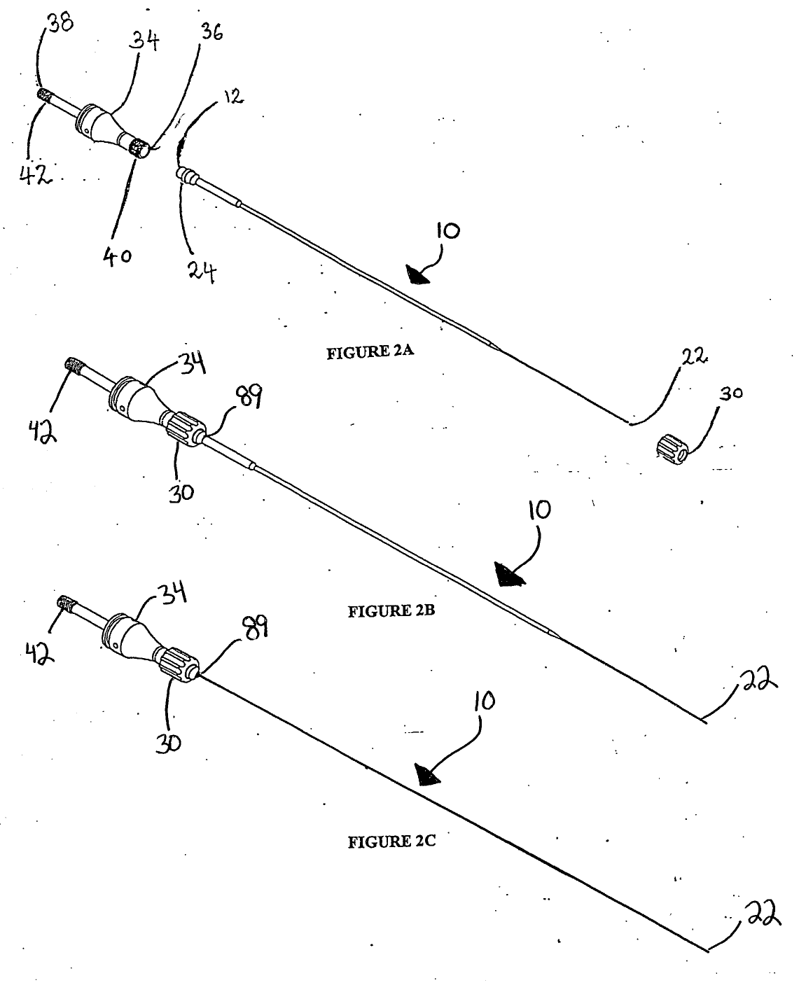

FIG. 2A and FIG. 2B show the unassembled and assembled views of individual components comprising the varied diameter probe, sound conductor elements, and the coupling assembly. FIG. 2A shows an elongated probe 10 and a horn assembly 34 comprising a proximal end 38 and a cylindrical slot 36 at the distal end. FIG. 2A also shows the horn, the coupling assembly components, the elongated probe 10, and the locking nut 30. The coupling assembly components comprise threading arrangements 40 and 42, a cylindrical slot 36, and a locking nut 30. Attachment of the proximal end 12 of the probe 10 is accomplished by insertion of the probe head 24 into the cylindrical slot 36 at the distal end of the horn assembly followed by “threading” the probe through the locking nut 30 to enable threads on the inner surface of the locking nut 30 (not shown) to engage a series of complementary threads of the threading arrangement 40. As such, an intimate contact is provided between the probe's proximal end 12 and the distal end of the horn assembly. The probe attachment is rendered to be mechanically rigid by tightening the locking nut 30.

FIG. 2B shows the elongated, varied diameter probe 10 attached to the horn assembly at a discontinuity 89 and held rigidly by the coupling assembly and maintaining an intimate contact between the “coupled” components. FIG. 2C shows a similar assembly comprising a constant, narrow diameter probe of the present invention.

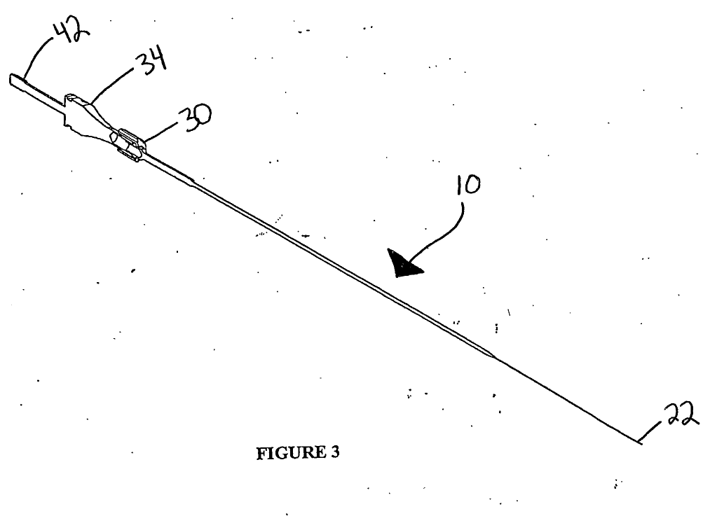

FIG. 3 shows a cross-sectional view of the probe-horn assembly shown in a “coupled” mode. The attachment means comprising the coupling assembly of the invention utilized to “couple” the elongated probe to the horn assembly is chosen from conventional means of connecting physically separated components in a manner so as to provide a rigid joining of said components while maintaining intimate material surface contact between the components in the “coupled” state. Suitable attachment means of the present invention include a locking nut comprising a screw thread, and a bayonet or ring clamp mechanism to effect coupling between the elongated probe and the horn assembly.

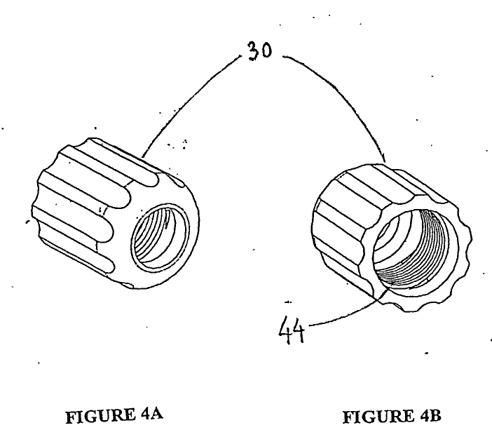

FIG. 4A and FIG. 4B show opposite-end views of a preferred embodiment of the locking means, comprising a locking nut 30 which comprises a screw thread arrangement 44 that is capable of engaging a complementary thread arrangement located along the outer diameter of the distal end of the horn assembly. When the horn assembly 34 is engaged with the elongated probe 10 and positioned proximally to provide “coupling”, the locking nut 30 provides a rigid interface between the probe and horn components and ensures intimate contact between the terminal end surfaces of the components; such coupling is important for efficient transmission of ultrasonic energy to the probe.

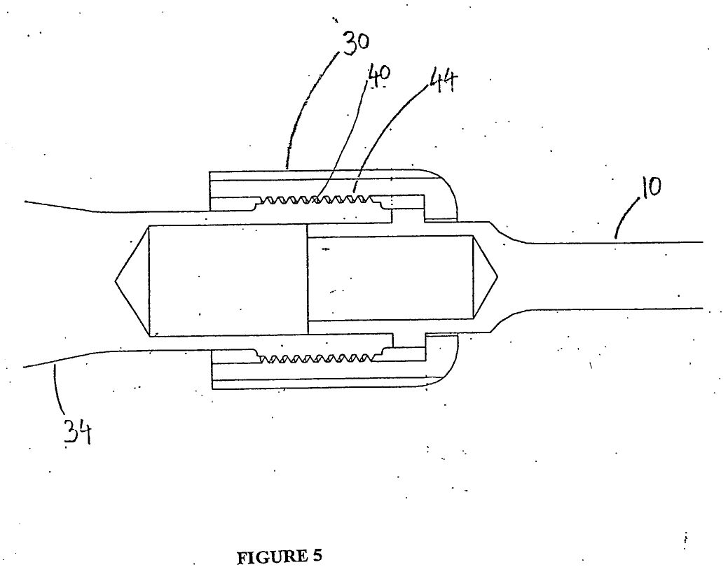

FIG. 5 shows a cross-sectional view of the horn assembly 34 and the elongated probe 10 “coupled” by the locking nut 30 of the invention by engaging the screw thread 44 with complementary threads 40 in the horn assembly.

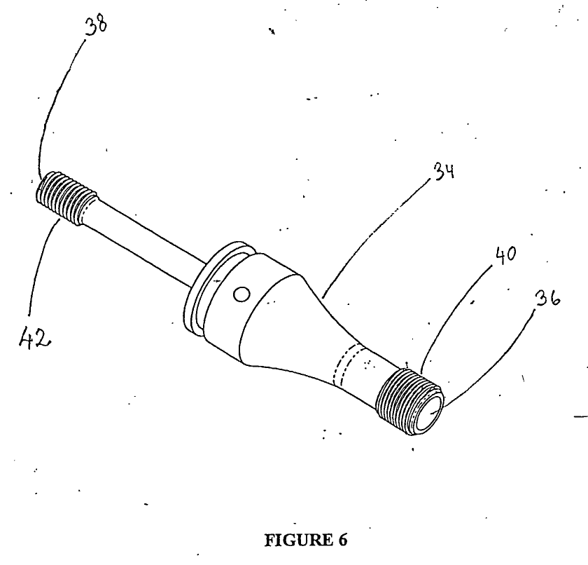

In FIG. 6, the horn assembly 34 comprises a cylindrical slot 36 at the distal end that is capable of being coupled to the elongated probe 10 of the invention, and a proximal end 38 of the horn assembly 34 that is coupled to a transducer (not shown), functioning as an ultrasonic energy source, by threading arrangements 40 and 42 located at either end. As mentioned previously, a horn assembly 34, comprising the sound conductor or “horn”, functions as an energy reservoir that allows only a small fraction of the energy transmitted by the source to the probe, thereby minimizing energy loss due to probe bending or damping that can occur when it is inserted into blood vessels.

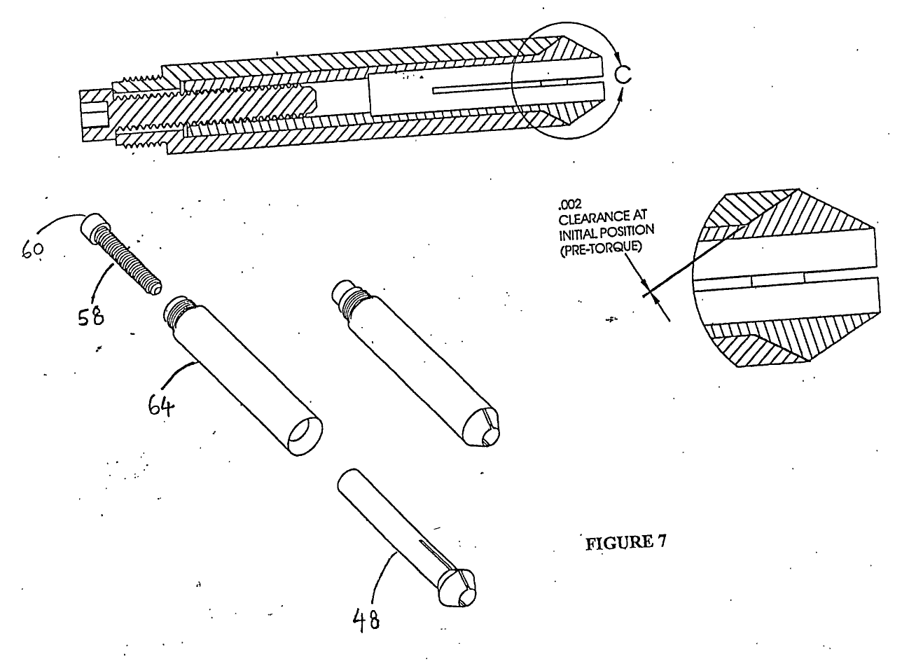

FIG. 7 shows disassembled and assembled views of another preferred embodiment of the probe attachment means of the invention. FIG. 7 shows cross-sectional views in the assembled state, that includes a coupling assembly comprising a “quick attachment/detachment” (QAD) collet rod 48 and a housing assembly 64 that enables efficient coupling of the elongated ultrasonic probe to the horn assembly (not shown). As seen in FIG. 7, a collet rod 48 is configured to slideably receive and retain the proximal end of the ultrasonic probe of the invention within the interior volume of the collet housing 64, and restrained in a rigid, non-removable manner by socket screw 58, which comprises a cylindrical head 60 with a uniformly flat end to facilitate its intimate contact with other device components, including the terminal end of the horn assembly.

FIG. 7 also shows regular and expanded cross-sectional views of the QAD collet rod 48 inserted into the collet housing 64 that is non-removably retained within the housing by a socket screw 58. As seen in segment “C” of the cross-sectional view, the inner surface of the collet housing tapers circumferentially outwardly at the distal end so as to enable partial insertion of the cylindrically slotted head of the QAD collet rod. The inner diameter of the circumferentially tapered section of the housing is chosen to be slightly larger then the insertable segment QAD collet rod head so as to create a “clearance” that facilitates easy insertion and retraction of the said collet rod (shown in the detail cross-sectional view in FIG. 7).

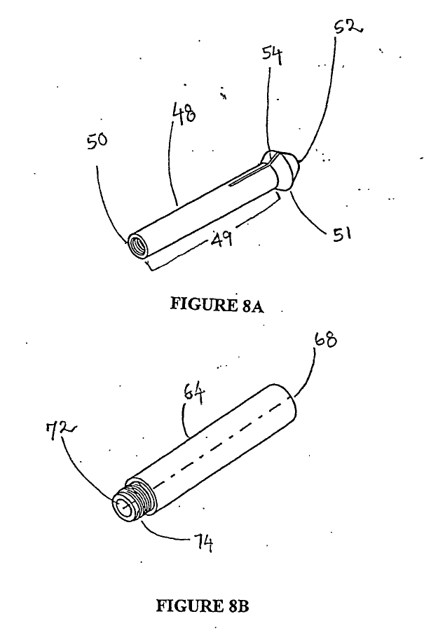

As shown in FIG. 8A, the QAD collet rod 48 is comprised of a hollow cylindrical segment 49 with a proximal end 50 and a head segment 51 at distal end 52 (the end farthest from the collet housing and horn assembly) with a diameter larger than that of the cylindrical segment. The head segment at the distal end 52 comprises a compressible slit 54 that is capable of accommodating the proximal end of the elongated probe. The proximal end 50 of the QAD collet rod comprises a hollow cylindrical opening containing a screw thread inscribed along the inner surface of said opening that is capable of receiving and retaining a socket screw 58 (shown in FIG. 7) inserted from the proximal end of the QAD collet housing, so as to render the collet rod 48 with the attached probe to be rigidly and non-removably restrained within said collet housing.

As shown in FIG. 8B, the collet housing 64 comprises a hollow cylinder with a distal end 68 capable receiving the cylindrical segment of the QAD collet rod 48 (FIG. 8A), and part of the cylindrically slotted head segment 51 when the collet rod is inserted at its proximal end 50 into collet housing 64. The collet housing 64 further comprises a proximal end 72 having a screw-thread 74 along the outer surface. The proximal end 72 of the collet housing further comprises a screw thread 74 on its outer surface capable of engaging the terminal end of a horn assembly in a manner so as to provide intimate contact between the horn and the flat head of socket screw 58 restraining QAD collet rod 48 attached to the elongated probe. The above-described structure enables transmission of ultrasonic energy from the horn to the elongated probe.

The socket screw 58 of the invention is capable of being “tightened” by applying a torque by conventional methods. Applying a torque causes the socket screw 58 to simultaneously engage the thread assemblies of the collet rod housing 64 and the QAD collet rod 48 respectively, after insertion of the collet rod into said housing. Such a tightening action which is performed after attachment of the elongated probe to the collet rod 48 by insertion of the probe into the compressible slit 54 at the distal end 52 of the collet rod causes retraction of the slotted head into the collet housing. This in turn, results in elimination of the “clearance” between the collet rod and the collet housing, causing a contraction in the diameter of the slot in the head of the collet rod and in turn, results in 1) its intimate contact with the surface of the proximal end of the inserted elongated probe, and 2) restraining the probe in a non-detachable manner to the collet rod—housing coupling assembly. The rigid and non-removable mode of probe attachment to the said coupling assembly enables transmission of ultrasonic energy from a horn assembly attached to the collet rod/housing coupling assembly to the elongated probe so as to cause it to vibrate in a transverse mode, and hence provide ultrasonic energy for tissue destruction. Conversely, the probe is detached (or “de-coupled”) from the collet rod/housing coupling assembly by loosening the socket screw 58 by application of a torque in a direction opposite to that used for the probe attachment process.

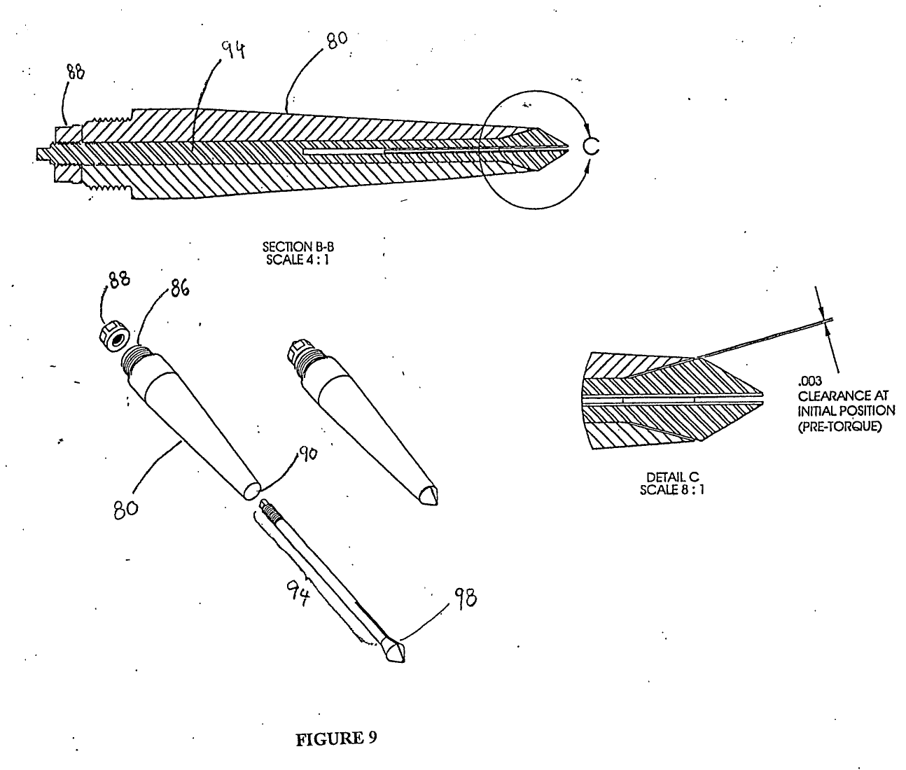

FIG. 9 shows disassembled and assembled views of another preferred embodiment of the probe attachment means of the invention. FIG. 9 shows cross-sectional views in the assembled state, comprising a QAD collet rod/housing assembly. The QAD collet rod/housing assembly comprises an outwardly cylindrically tapered collet housing component 80 with a proximal end 86 and a distal end 90, further comprising a centrally located cylindrical bore with open ends extending through its longitudinal axis that is capable of slideably receiving and retaining a collet rod. As seen in segment “C” of the cross-sectional view in FIG. 9, the inner surface of the collet housing tapers circumferentially outwardly at the distal end so as to enable partial insertion of the cylindrically slotted head of the QAD collet rod. The inner diameter of the circumferentially tapered section of the housing is chosen to be slightly larger then the insertable segment of the QAD collet rod head so as to create a “clearance” that facilitates easy insertion and retraction of the said collet rod (shown in the detail cross-sectional view). The cross-sectional view of FIG. 9 shows the QAD collet rod restrained within the collet rod housing by a locking nut 88.

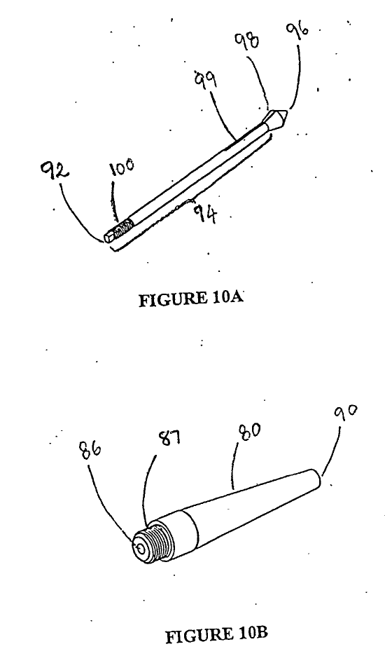

FIG. 10A and FIG. 10B show the collet rod and collet housing respectively. As seen in FIG. 10A, the QAD collet rod comprises a solid cylindrical body 94 with a head segment 98 attached at the distal end 96. A longitudinal slit 99 extends from the head segment 98 partially into the cylindrical body 94. The proximal end 92 of the cylindrical body 94 comprises a thread assembly 100.

As seen in FIG. 10B, the collet housing 80 comprises a cylindrical rod with a continuously decreasing external diameter from the proximal end 86 to the distal end 90, further comprising a centrally located cylindrical inner bore extending along its entire length providing openings at both ends. The diameter of the bore decreases from the proximal end to the distal end so as to circumferentially taper outwardly in a manner permitting partial insertion of the head segment 98 of the collet rod. The cylindrical bore of the collet housing 80 is capable of slideably receiving a collet rod 94 such that the thread assembly 100 of the said collet rod extends beyond the proximal end 86 of the housing assembly 80 to permit a rigid and non-removable attachment of the collet rod by engaging the thread assembly 100 with the locking nut 88 (shown in FIG. 9). The locking nut performs a similar function and in a manner that is substantially similar to that of the restraining screw described in a previous embodiment (FIG. 7) in enabling the elongated probe to be non-removably attached to and detached from the QAD collet rod for operation of the device as previously described. Upon rigid non-removable attachment of the elongated probe to the coupling assembly, the threading 87 of the collet housing is engaged to complementary threading of the horn assembly (not shown) so as to render intimate contact of the sound conductor (horn) in said horn assembly with the proximal end 92 of the collet rod to enable transmission of ultrasonic energy from the horn to the elongated probe attached at distal end 96 of the collet rod.

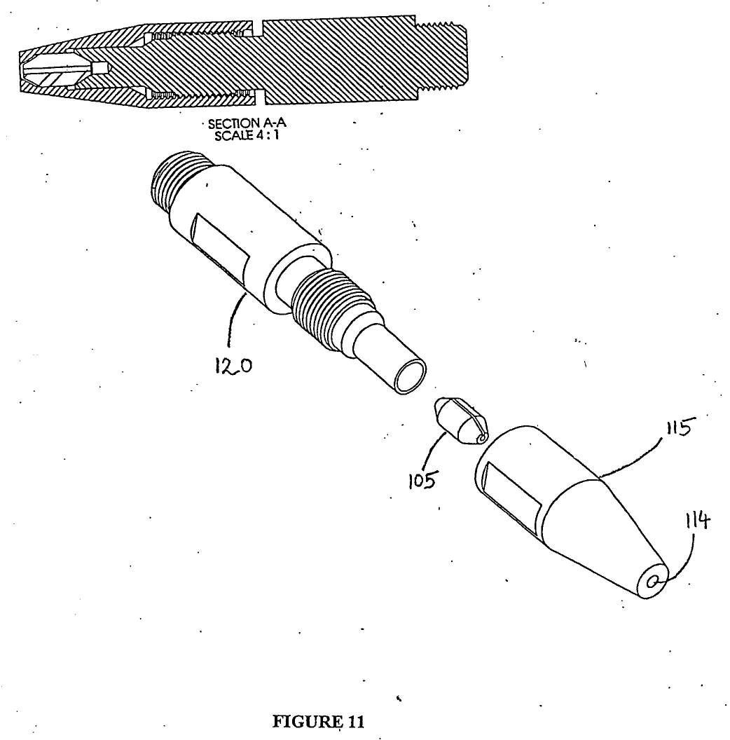

FIG. 11 shows a preferred embodiment of probe coupling assembly of the invention, including a cross-sectional view, comprising a QAD collet 105 that is insertable into a “compression” collet housing component 115 comprising a circular bore 114 that is detachably connected to a QAD base component 120.

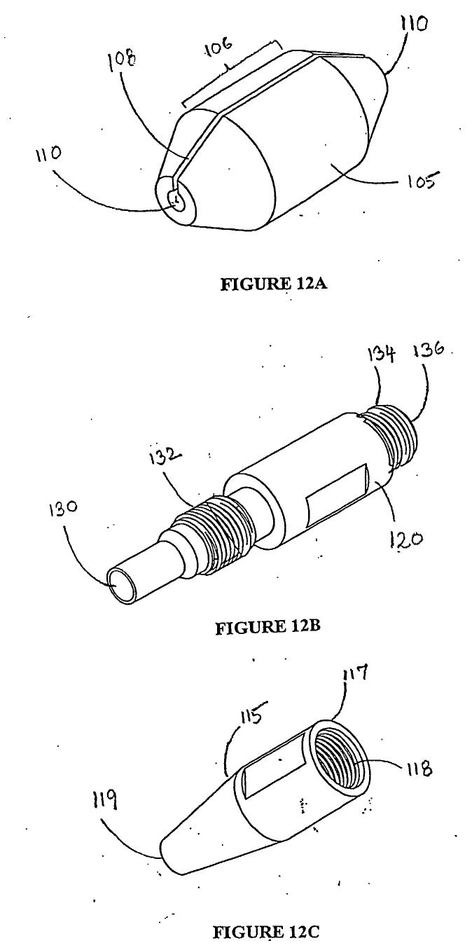

As seen in FIG. 12A, the QAD collet 105 comprises a cylindrical segment 106 with a cylindrical slot 108 extending through its longitudinal axis that is capable of slideably receiving the proximal end of the elongated probe and it is symmetrically tapered at the proximal and the distal end 110.

As seen in FIG. 12B, QAD base component 120 comprises a conical slot 130 at the cylindrical distal end capable of accommodating one of the symmetrically tapered ends 110 of the collet. The QAD base component 120 further comprises a thread assembly 132 located along its outer circumference near its distal end, that is capable of engaging complementary threads in the QAD compression collet housing component 115. The proximal end 136 of the base component contains a thread assembly 134 along the outer circumference that is capable of engaging and attaching to the horn assembly (not shown) of the invention.

As seen in FIG. 12C, the QAD compression collet housing component 115 comprises a hollow cylindrical segment with a proximal end 117 and a circular bore 114 (shown in FIG. 11); the QAD compression collet housing component further comprises a tapered distal end 119 capable of slideably receiving the proximal end of the elongated probe. The inner diameter at the proximal end of the QAD compression housing component 115 is chosen so as to accommodate the symmetrically tapered terminal end 110 of the collet 105 that is distal to the base component, and further comprises a thread assembly 118 that enables the compression housing component to engage a series of complementary threading 132 on the distal end of QAD base component 120. The proximal end of the elongated probe of the invention is inserted through the circular bore 114 at the distal end of compression housing component 115 and the symmetrically tapered end 110 of the collet 105 is inserted in a manner so as to occupy the entire length of the cylindrical slot 108 inside the collet 105. The other symmetric end 110 distal to the compression housing 115 is then placed inside a conical pocket 130 of the base component 120, following which threads 118 of the compression housing is engaged with the complementary threads 132 in the QAD base component 120 by applying a torque so as to render the collet 105 to be non-removably retained inside the coupled base-compression housing assembly; the probe is thereby restrained rigidly and non-removably within the coupling assembly. Additionally, the mode of restraint provided by the coupling assembly of the embodiment enables the probe to maintain an intimate contact with said assembly and in turn the horn assembly (not shown) of the invention is attached to the coupling assembly by engaging a thread 134 in the QAD base component 120 with complementary threading in the horn assembly. Ultrasonic energy transmitted from the horn is therefore communicated to the probe via the coupling assembly. The elongated probe is detached by disassembling the coupling assembly, thereby allowing the probe to be withdrawn from the collet 105 and compression housing component 115.

Upon being activated, the device of the present invention causes the ultrasonic energy generator component to transmit ultrasonic energy to the horn component. The transmitted energy is amplified by the horn component, which in turn, due to it's intimate and proximal contact with the elongated probe, transmits the amplified energy to the probe. Transverse vibration modes on the elongated probe that fall within the horn resonance are therefore, excited. The “coupling” between the elongated probe and the horn is configured so to as to present a relatively large impedance mismatch. In one embodiment of the present invention, the coupling is located at an anti-node of the horn. In one embodiment, the coupling is located at a node of the horn. Longitudinal waves impinging on the coupling will be either reflected back inside the horn, or transmitted outward to the elongated probe proportionally to the degree of the impedance mismatch at the coupling interface. In a preferred embodiment, the coupling is arranged in a manner so as to cause reflection of a substantial portion of the ultrasonic energy back into the horn. Under these conditions, the horn essentially functions as an energy storage device or reservoir, thereby allowing for a substantial increase in drive amplitude.

The ultrasonic device of the present invention provides several advantages for tissue ablation within narrow arteries over prior art devices. The transverse energy is transmitted extremely efficiently, and therefore the required force to cause cavitation is low. The transverse probe vibration provides sufficient cavitational energy at a substantially low power (˜1 watt). Ultrasonic energy is supplied to surrounding tissue along the entire length of the probe as opposed to solely at the probe tip, the rates of endovascular materials that can be removed are both significantly greater and faster as compared to prior art devices. The transverse vibrational mode of the elongated probe and the attachable/detachable coupling mode to the horn assembly allows for the bending of the probe without causing damage to the probe or damage to the surrounding tissue.

Another advantage offered by the device of the present invention is the innovative mechanism for probe attachment and detachment by means of a lateral wall compression and decompression provided by the coupling assembly. The probe can therefore, be rapidly attached to and detached from the coupling assembly without necessitating the traditional “screwing” or “torquing” that are utilized with prior art methods of attaching an ultrasonic probe to a probe handle. This feature facilitates ease of manipulation of the probe within narrow and torturous venal cavities, and its positioning at the occlusion site in a manner substantially similar to narrow lumen catheters prior to and after device use.

All references, patents, patent applications and patent publications cited herein are hereby incorporated by reference in their entireties. Variations, modifications, and other implementations of what is described herein will occur to those of ordinary skill in the art without departing from the spirit and scope of the present invention as claimed. Accordingly, the present invention is to be defined not by the preceding illustrative description but instead by the spirit and scope of the following claims.

What is claimed is:

1. A device for treating occlusions in a body comprising:
a probe having a proximal end and a distal end;
a horn having a first connection end and a second connection end wherein the first connection end engages the proximal end of the probe;
a handle engaging the second connection end of the horn; and
a discontinuity at a point of attachment where the probe engages the horn wherein the discontinuity creates an impedance mismatch between the probe and the horn.

2. The device of claim 1 wherein a diameter of the probe is approximately 0.025 inches or less.

3. The device of claim 1 wherein a diameter of the probe varies from the proximal end to the distal end of the probe.

4. The device of claim 1 wherein the handle is capable of delivering ultrasonic energy from the handle to the probe.

5. The device of claim 1 wherein the probe oscillates in a transverse mode.

6. The device of claim 1 wherein the horn is a mason horn.

7. The device of claim 1 wherein a length of the horn approximates an integer multiple of one-half wavelength of a vibration.

8. The device of claim 1 wherein the horn is a longitudinal drive system.

9. The device of claim 1 wherein the horn is a transverse drive system.

10. The device of claim 1 wherein the discontinuity is placed at a location of an anti-node along the probe.

11. The device of claim 1 wherein the discontinuity is placed at a location of a node along the probe.

12. The device of claim 7 wherein the length of the horn is increased by approximately one-fourth of a wavelength when the discontinuity is placed at a location of a node along the probe.

13. The device of claim 12 wherein a second discontinuity is placed approximately one-fourth of a wavelength away from the discontinuity at the point of attachment of the probe and the horn.

14. The device of claim 1 wherein the discontinuity is created by a significant decrease in a diameter between the probe and the horn at the point of attachment.

15. The device of claim 1 wherein the discontinuity is created by a dense material comprising the horn and a less dense material comprising the probe.

16. The device of claim 1 wherein the horn is comprised of aluminum or an aluminum alloy.

17. The device of claim 1 wherein the horn is comprised of steel or a ferrous material.

18. The device of claim 1 wherein the probe is comprised of titanium or a titanium alloy.

19. The device of claim 1 wherein the discontinuity is created by using a horn comprised of a first material having a first elastic modulus and a probe comprised of a second material having a second elastic modulus wherein the first elastic modulus and the second elastic modulus are different.

20. The device of claim 1 wherein the discontinuity results in a return of approximately 80 percent of the ultrasonic energy generated in the handle back into the horn and a transfer of the remaining approximately 20 percent of the ultrasonic energy into the probe.

21. The device of claim 1 wherein the horn is independent of a vibrational motion of the probe.

22. A device for removing occlusions in a blood vessel comprising:
an ultrasonic probe comprising a proximal end and a distal end;
a sound conductor comprising a proximal end and a distal end, wherein the distal end of the sound conductor is engaged to a coupling assembly and the proximal end of the sound conductor is engaged to a transducer capable of providing ultrasonic energy; and
a discontinuity between the ultrasonic probe and the sound conductor at a point of attachment between the ultrasonic probe and the sound conductor,
wherein the ultrasonic probe is releasably mounted at the proximal end of the ultrasonic probe to the coupling assembly, enabling the sound conductor to transmit ultrasonic energy from the transducer to the ultrasonic probe, causing the ultrasonic probe to oscillate in a substantially transverse mode with respect to a longitudinal axis of the ultrasonic probe.

23. The device of claim 22 wherein the ultrasonic probe is a flexible, elongated wire.

24. The device of claim 22 wherein a diameter of the ultrasonic probe varies along the longitudinal axis of the ultrasonic probe.

25. The device of claim 22 wherein the flexural stiffness of the ultrasonic probe varies along the longitudinal axis of the ultrasonic probe.

26. The device of claim 22 wherein a diameter of the ultrasonic probe remains constant along the longitudinal axis of the ultrasonic probe.

27. The device of claim 22 wherein a length of the ultrasonic probe is between approximately 30 centimeters and approximately 300 centimeters.

28. The device of claim 22 wherein the ultrasonic probe further comprises a sheath assembly adapted to the ultrasonic probe that includes at least one sheath.

29. The device of claim 28 wherein the sheath assembly substantially prevents transmission of cavitational energy generated by the ultrasonic probe to a surrounding environment.

30. The device of claim 28 wherein the sheath assembly further comprises at least one fenestration in the at least one sheath.

31. The device of claim 30 wherein the fenestration in the at least one sheath is capable of transmitting cavitational energy therethrough to the surrounding environment.

32. The device of claim 28 wherein the sheath assembly further comprises at least one reflective element.

33. The device of claim 28 wherein the sheath assembly further comprises at least one irrigation channel.

34. The device of claim 28 wherein the sheath assembly further comprises at least one aspiration channel.

35. The device of claim 28 wherein the sheath assembly further comprises at least one channel for delivering a therapeutic agent therethrough.

36. The device of claim 28 wherein the sheath assembly further comprises an imaging system.

37. The device of claim 28 wherein the sheath assembly is adapted for use with an imaging system.

38. The device of claim 28 wherein the sheath assembly is a vascular catheter comprising at least one lumen.

39. The device of claim 22 wherein the coupling assembly is capable of connecting the probe to the sound conductor and a transducer capable of vibrating at an ultrasonic frequency.

40. The device of claim 22 wherein the sound conductor and the transducer are contained in a handle of the device.

41. The device of claim 22 wherein the coupling assembly comprises a releasable compressive clamp mounted externally to a collet residing in a housing assembly at the distal end of the coupling assembly, the collet capable of releasably engaging the ultrasonic probe.

42. The coupling assembly of claim 41 wherein the releasable compressive clamp is capable of exerting a compressive force on the collet causing the collet to engage the ultrasonic probe.

43. The device of claim 22 wherein the coupling assembly enables attachment and detachment of the ultrasonic probe.

44. The device of claim 22 wherein the sound conductor engaged to the coupling assembly is capable of controlling ultrasonic energy transferred to the ultrasonic probe.

45. The device of claim 22 wherein the horn is independent of a vibrational motion of the probe.

46. A method of delivering an ultrasonic energy to a region in need of a treatment inside of a body comprising:
decoupling a drive system from an ultrasonic probe by placing a discontinuity at a point where the drive system engages the ultrasonic probe wherein the drive system operates at a predictable frequency which is unaffected by changes in the frequency of the probe;
positioning the ultrasonic probe to the region in need of treatment inside of the body; and
delivering the ultrasonic energy to the region in need of treatment.

47. The method of claim 46 wherein decoupling occurs by providing a discontinuity at a point of attachment where the probe is attached to a horn such that the discontinuity creates an impedance mismatch between the probe and the horn.

48. The method of claim 46 wherein a diameter of the probe is approximately 0.025 inches or less.

49. The method of claim 46 wherein a diameter of the probe varies from the proximal end to the distal end.

50. The method of claim 46 wherein a handle is capable of delivering an amount of ultrasonic energy from the probe to the handle.

51. The method of claim 46 wherein the probe oscillates in a transverse mode.

52. The method of claim 46 further comprising engaging the drive system to the ultrasonic probe by a horn.

53. The method of claim 52 wherein the horn is a mason horn.

54. The method of claim 52 wherein a length of the horn is approximates an integer multiple of one-half wavelength of a vibration.

55. The method of claim 52 wherein the horn is a longitudinal drive system.

56. The method of claim 52 wherein the horn is a transverse drive system.

57. The method of claim 46 wherein the discontinuity is placed at an anti-node location along the probe.

58. The method of claim 46 wherein the discontinuity is placed at a node location along the probe.

59. The method of claim 54 wherein the length of the horn is increased by approximately one-fourth of a wavelength away from the discontinuity at the point of attachment of the drive system and the horn.

60. The method of claim 52 wherein the discontinuity is created by a significant change in diameter of the device at the point of attachment between the probe and the horn.

61. The method of claim 52 wherein the discontinuity is created by a change in the density of the device at the point of attachment between the probe and the horn.

62. The method of claim 52 wherein the horn is comprised of aluminum or an aluminum alloy.

63. The method of claim 52 wherein the horn is comprised of steel or a ferrous material.

64. The method of claim 46 wherein the probe is comprised of titanium or a titanium alloy.

65. The method of claim 52 wherein the discontinuity is created by using the horn comprised of a first material comprising a first elastic modulus and a probe comprised of a second material comprising a second elastic modulus wherein the the first elastic modulus and the second elastic modulus are different.

66. The method of claim 46 wherein the discontinuity results in a return of approximately 80 percent of the ultrasonic energy generated in the handle back into the horn and a transfer of the remaining approximately 20 percent of the ultrasonic energy into the probe.

67. The method of claim 46 wherein the drive system is independent of a vibrational motion of the probe.

68. A method of removing occlusions in a blood vessel using an ultrasonic device comprising the following steps:
(a) inserting an ultrasonic probe into the site of an occlusion in a body;
(b) positioning the ultrasonic probe in the proximity of the occlusion by an axial or rotational manipulation within the occluded blood vessel;
(c) mounting the ultrasonic probe to a coupling assembly;
(d) activating the transducer to cause oscillation of the ultrasonic probe in a substantially transverse mode with respect to a longitudinal axis of the probe;
(e) decoupling a drive system from the ultrasonic probe wherein the drive system operates at a predictable frequency which is unaffected by changes in the frequency of the probe; and
(f) providing ultrasonic energy to the ultrasonic probe to remove occlusions.

69. The method of claim 68 wherein the ultrasonic probe is a flexible, elongated guidewire.

70. The method of claim 68 wherein the ultrasonic probe further comprises a sheath assembly comprising at least one sheath.

71. The method of claim 70 wherein the sheath is capable of partially shielding a tissue from the ultrasonic probe at the site of the occlusion.

72. The method of claim 70 wherein the sheath assembly comprises an aspiration conduit, whereby fragments of an occlusive material are removed through the conduit.

73. The method of claim 72 wherein the sheath assembly further comprises an irrigation conduit wherein the irrigation conduit enables a supply of an irrigation fluid to the site of treatment in order to facilitate the removal of an occlusive material.

74. The method of claim 70 wherein the sheath assembly comprises a conduit for delivering a therapeutic agent through the conduit and to the treatment site.

75. The method according to claim 70 wherein the sheath assembly is a vascular catheter comprising at least one lumen.

76. The method of claim 68 wherein the drive system is independent of a vibrational motion of the probe.
